(19) United States
(12) Patent Application Publication
(10) Pub. No.: US 2011/0125206 A1
(43) Pub. Date: May 26, 2011

(54) SINGLE CHAMBER IMPLANTABLE MEDICAL DEVICE FOR CONFIRMING ARRHYTHMIA THROUGH RETROSPECTIVE CARDIAC SIGNALS

(75) Inventors: Gene A. Bornzin, Simi Valley, CA (US); Jong Gill, Valencia, CA (US); Jeffery D. Snell, Chatsworth, CA (US)

(73) Assignee: PACESETTER, INC., Sylmar, CA (US)

(21) Appl. No.: 12/625,416

(22) Filed: Nov. 24, 2009

(51) Int. Cl. A61N 1/39; A61B 5/0452; A61B 5/046

(52) U.S. Cl. 607/5; 600/515; 600/518

(57) ABSTRACT

An implantable medical device is provided that comprises a housing, sensors configured to be located to proximate a heart, and a sensing module to sense cardiac signals originating from the heart over a channel defined by the sensors. The cardiac signals include intrinsic R-wave events and associated intrinsic confirmation events when the heart exhibits normal sinus rhythm. The device further includes memory to store the cardiac signals sensed over a channel, and a detection module. The detection module identifies an R-wave event within the cardiac signals. The detection module captures, in the memory, a segment of the cardiac signals that precedes the R-wave event as a retrospective segment. The detection module determines whether the retrospective segment includes an intrinsic confirmation event that is associated with and occurs before the R-wave event. The detection module declares an arrhythmia based at least in part on the determination of whether the retrospective segment includes the intrinsic confirmation event is absent from the retrospective segment.

### FIELD OF THE INVENTION

The present invention generally relates to the field of implantable medical devices. Embodiments more particularly relate to implantable medical devices that confirm arrhythmias based on retrospective cardiac signals collected preceding a current event of interest.

### BACKGROUND OF THE INVENTION

An implantable medical device (IMD) is well known in the art. The IMD may take the form of implantable defibrillators or cardioverters which treat accelerated rhythms of the heart such as fibrillation. The IMD may also take the form of implantable pacemakers which maintain the heart rate above a prescribed limit, such as, for example, to treat a bradycardia. Implantable cardiac devices are also known which incorporate both a pacemaker and a defibrillator. As a further example, the IMD may be an implantable monitoring device, such as the Confirm™ device offered by St. Jude Medical.

An IMD is comprised of three major components. One component, at least in stimulation type IMDs, is a pulse generator which generates the stimulation pulses and includes the electronic circuitry and the power cell or battery. The second component, at least in stimulation type IMDs, is the lead, or leads, which electrically couple the IMD to the heart. IMDs deliver stimulation pulses to the heart to cause the stimulated heart chamber to contract when the patient's own intrinsic rhythm fails. The third component is a sensor and detection module that monitors a heart for cardiac signals and analyzes the cardiac signals to identify normal sinus rhythm, arrhythmias and the like. To this end, IMDs include sensing circuits that sense cardiac activity for the detection of intrinsic cardiac events such as intrinsic atrial events (P-waves) and intrinsic ventricular events (R-waves). By monitoring P-waves and/or R-waves, the IMD circuits are able to determine the intrinsic rhythm of the heart and provide stimulation pulses that force atrial and/or ventricular depolarizations at appropriate times in the cardiac cycle when required to help stabilize the electrical rhythm of the heart.

Many stimulation type IMDs are described as single-chamber or dual-chamber systems. A single-chamber system stimulates and senses in the same chamber of the heart (atrium or ventricle). A dual-chamber system stimulates and/or senses in both chambers of the heart (atrium and ventricle). Dual-chamber systems may typically be programmed to operate in either a dual-chamber mode or a single-chamber mode. Further, IMD systems are known which deliver stimulation pulses at multiple sites. For example, biventricular pacing paces in both ventricles and biatrial pacing paces in both atria. Hence, it is possible, that a heart may be stimulated in all four chambers.

Atrial fibrillation involves an abnormality of electrical impulse formation and conduction that originates in the atria. Atrial fibrillation is characterized by multiple swirling wavelets of electrical current spreading across the atria in a disorganized manner. The irregularity of electrical conduction throughout the atria creates irregular impulse propagation through the atrioventricular (AV) node into the ventricle.

Traditionally, single chamber IMDs detect atrial fibrillation (AF) based on one or more of ventricular rate, rate stability, and the morphology of the cardiac signal. However, conventional algorithms for detecting AF experience certain limitations. For example, conventional AF detection algorithms, that are based on rate stability, may become confounded when an atrial tachyarrhythmia drives a ventricle at a high, but very stable rate. When a patient experiences atrial tachyarrhythmia having a stable rate, the AF detection algorithm may classify the events merely as high rate normal sinus events. Thus, the AF detection algorithm may not declare the events to be pathologic (non-physiologic) and may not deliver a therapy.

Moreover, conventional morphology detection algorithms may not correctly classify atrial fibrillation that exhibits rate dependent changes in the QRS complex. When a patient experiences atrial tachyarrhythmia having rate dependent changes in the QRS complex, the morphology detection algorithm may classify the events merely as physiologic events and thus, may not declare the events to be pathologic.

Furthermore, conventional AF detection algorithms that are based on rate stability may not identify a premature ventricular contraction (PVC) that occurs during AF. The PVC during normal sinus rhythm may mimic a cardiac signal having rate instability during AF thus leading to producing false diagnosis of AFG detection. The above examples of certain types of events may lead to a failure to correctly diagnosis atrial fibrillation. Also, the above exemplary event types may conversely be falsely classified as a ventricular tachyarrhythmia (VT). When a VT is falsely classified, the IMD may deliver a high energy cardioversion pulse when not needed, which is not desirable. The above uncertainties reduce the confidence in AF detection algorithms used by current implantable medical devices. Because of the low confidence in AF detection, conventional single chamber IMDs do not provide any form of AF burden diagnostic.

Moreover, misclassification of AF has serious ramifications since AF is treated with anticoagulation drugs. The decision to place a patient on anticoagulation therapy is not taken lightly. Certain anticoagulation drugs may have side effects and require frequent blood sampling, such as to assure that the INR (International Normalized Ratio) is in the therapeutic range. Therefore, a highly sensitive and specific diagnostic for arrhythmia's, such as AF with IEGM confirmation would be highly desirable in single chamber IMDs.

A need remains for a much more sensitive and specific arrhythmia detection and confirmation method and system. A need remains for an implantable medical device that detects and confirms arrhythmias, such as atrial fibrillation, that drive the ventricle at a high, but stable rate.

### SUMMARY OF THE INVENTION

In accordance with one embodiment, an implantable medical device is provided that comprises a housing, sensors configured to be located to proximate a heart, and a sensing module to sense cardiac signals originating from the heart over a channel defined by the sensors. The cardiac signals include intrinsic R-wave events and associated intrinsic confirmation events when the heart exhibits normal sinus rhythm. The device further includes memory to store the cardiac signals sensed over the channel and a detection module. The detection module identifies an R-wave event within the cardiac signals. The detection module captures, in the memory, a segment of the cardiac signals sensed that precedes the R-wave event as a retrospective segment. The detection module determines whether the retrospective segment includes an intrinsic confirmation event that is associated with and occurs before the R-wave event. The detection module declares an arrhythmia based at least in part on the determination of whether the intrinsic confirmation event is absent from the retrospective segment.

Optionally, a lead may be configured to be coupled to the housing and to be located proximate to a heart. The lead includes at least a portion of the sensors utilized to define a primary and a secondary channel. Optionally, the detection module identifies the R-wave event from a near field signal component and analyzes a far field signal component for the intrinsic confirmation event. The sensing module may utilize different sets of sensors to sense cardiac signals over primary and secondary channels. The sensing module may include high and low sensitivity amplifiers coupled to the sensors. The sensing module may utilize a common set of sensors to define the primary and secondary channels, where the primary channel is coupled to the low sensitivity amplifiers, and the secondary channel is coupled to the high sensitivity amplifiers.

In at least one embodiment, the intrinsic confirmation event is one of a P-wave, a HIS signal and a PVC signal. In one embodiment, the detection module declares the arrhythmia which constitutes one of atrial fibrillation, flutter, and bigeminy. Optionally, the sensing module may monitor a near field (NF) channel for the R-wave event originating within a chamber of the heart proximate to a first sensor and monitor a far field (FF) channel for the intrinsic confirmation event originating within a chamber of the heart remote from the sensor. Optionally, the detection module may identify the R-wave event over multiple cardiac cycles. The detection module identifies the arrhythmia based at least in part on the degree of correlation. The sensors may include at least one of an RV shocking coil, RV ring, an SVC coil and RV tip, the sensing module defining a secondary channel between one of the SVC and the housing and one of the RV shocking coil, RV ring, and RV tip.

In an alternative embodiment, a method is provided for confirming an arrhythmia in an implantable device. The method comprises sensing cardiac signals originating from the heart over a channel defined by the sensors that are configured to be located to proximate a heart; and storing, in memory in the implantable device, the cardiac signals sensed, the cardiac signals include intrinsic R-wave events and associated intrinsic confirmation events when the heart exhibits normal sinus rhythm. The method further includes identifying an R-wave event within the cardiac signals sensed over the primary channel. When the R-wave event is identified, the method includes capturing a segment of the cardiac signals sensed that precedes the R-wave event as a retrospective segment. The method further includes determining whether the retrospective segment includes an intrinsic confirmation event that is associated with and occurs before the R-wave event; and declaring an arrhythmia based at least in part on the determination of whether the intrinsic confirmation event is absent from the retrospective segment.

In accordance with one embodiment, upon detection of each R-wave, the far field ventricular electrogram is scanned backwards in time, such as for about 350 ms to verify the presence of a P-wave. Although the P-wave may be relatively low in amplitude, the high performance, low noise, digital systems in the device 10 may be used to provide high confidence in P-wave detection. The P-wave template may be acquired automatically by performing ensemble averaging of the far field P-waves. The P-wave template is then cross-correlated in the buffered, retrospective window preceding each R-wave. A match indicates the presence of a P-wave. Consistent detection of a far-field P-wave prior to an R-wave positively identifies the presence of normal sinus rhythm. Conversely, near absence or inconsistent detection of a P-wave indicates an arrhythmia.

### BRIEF DESCRIPTION OF THE DRAWINGS

Further features and advantages of the present invention may be more readily understood by reference to the following description taken in conjunction with the accompanying drawings, in which:

FIG. 1 is a simplified diagram illustrating an implantable stimulation device in electrical communication with a lead implanted into a patient's heart for detecting arrhythmias, and delivering stimulation and shock therapy.

FIG. 2 is a functional block diagram of an implantable medical device formed in accordance with an embodiment of the present invention.

FIG. 3 is a diagram illustrating a cardiac cycle exhibiting normal physiologic behavior.

FIG. 4 is a diagram illustrating a cardiac cycle exhibiting an arrhythmia.

FIG. 5 is a flow chart describing an arrhythmia detection operation in accordance with one embodiment of the present invention.

FIG. 6 illustrates a graphical representation of a process carried out in connection with capturing and combining retrospective segments in accordance with one embodiment of the present invention.

FIG. 7 illustrates an implantable monitoring device intended for subcutaneous implantation at a site near the heart.

FIG. 8 illustrates a distributed processing system in accordance with one embodiment of the present invention.

### DESCRIPTION OF THE PREFERRED EMBODIMENTS

This description is not to be taken in a limiting sense but is made merely for the purpose of describing the general principles of the invention. The scope of the invention should be ascertained with reference to the issued claims. In the description of the invention that follows, like numerals or reference designators will be used to refer to like parts or elements throughout.

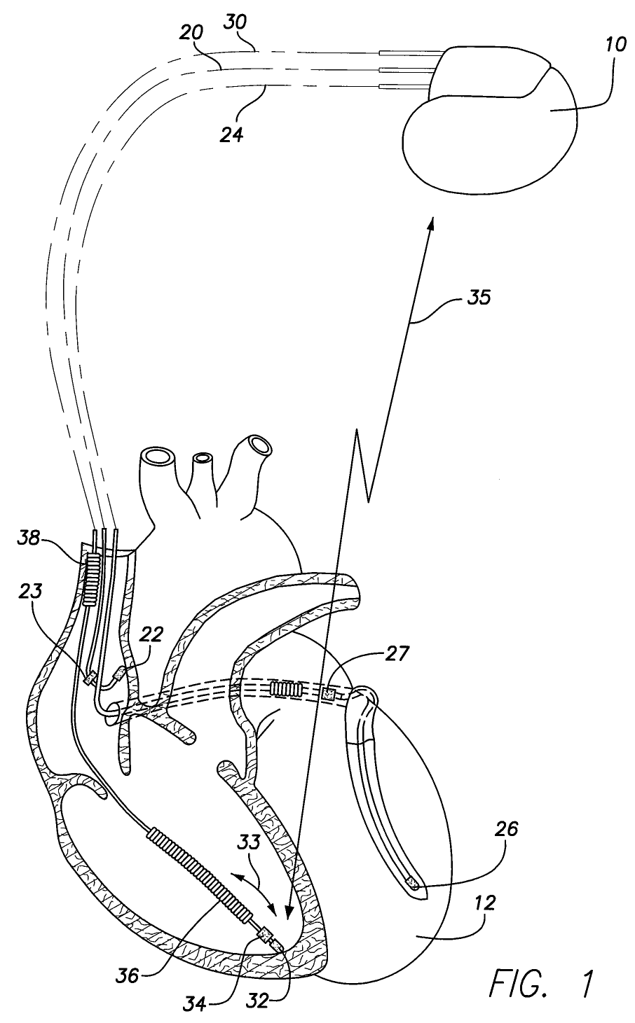

FIG. 1 illustrates a simplified block diagram of a dual-chamber implantable stimulation device 10, which is capable of treating both fast and slow arrhythmias with stimulation therapy, including cardioversion, defibrillation, and pacing stimulation. To provide atrial chamber pacing stimulation and sensing, the stimulation device 10 is shown in electrical communication with a patient's heart 12 by way of an implantable unipolar atrial lead 20 having an atrial tip electrode 22 implanted in the patient's atrial appendage and an atrial ring electrode 23. The stimulation device 10 is also in electrical communication with the patient's heart 12 by way of an implantable RV lead 30 having, in this embodiment, a RV tip electrode 32, an RV ring electrode 34, a RV (RV) coil electrode 36, and a superior vena cava (SVC) coil electrode 38. Typically, the ventricular lead 30 is transvenously inserted into the heart 12 so as to place the RV coil electrode 36 in the RV apex, and the SVC coil electrode 38 in the superior vena cava. Accordingly, the ventricular lead 30 is capable of receiving cardiac signals, and delivering stimulation in the form of pacing and shock therapy to the right ventricle. The stimulation device 10 is also in electrical communication with the patient's heart 12 by way of an implantable LV lead 24 having, in this embodiment, an LV tip electrode 26 and an LV ring electrode 27. Typically, the LV lead 24 is transvenously inserted into the Coronary Veins of the heart 12. Accordingly, the LV lead 24 is capable of receiving cardiac signals, and delivering stimulation in the form of pacing and shock therapy to the left ventricle. Additionally, an LV coil may be provided.

The leads 30, 20 and 24 may represent unipolar leads or bipolar leads, and may be used in a unipolar sensing mode or a bipolar sensing mode. Also, although three leads are shown in FIG. 1, it should also be understood that fewer or additional stimulation leads (with one or more pacing, sensing and/or shocking electrodes) may be used in order to efficiently and effectively provide pacing stimulation to the left side of the heart or atrial cardioversion and/or defibrillation. For example, a lead designed for placement in the coronary sinus region could be implanted to deliver left atrial pacing and atrial or ventricular shocking therapy.

When in the unipolar sensing mode, a sense amplifier detects electrical voltage differentials between a single electrode and the external body or housing of the IMD 10. In a dual-chamber, dual-unipolar pacing system, one unipolar lead is inserted within the atria and another within the ventricles, from which the device derives separate atrial and ventricular channel EGM signals. When in a bipolar sensing mode, a bipolar lead includes two electrodes mounted in close proximity to one another within the heart, such as tip and ring electrodes. One bipolar lead may be inserted within the atria and another within the ventricles, from which the device derives separate atrial and ventricular signals. An atrial sense amplifier detects electrical voltage differentials between the tip and ring electrodes of the atrial lead.

As explained hereafter, the device 10 processes and filters sensed cardiac signals to separate near field signals and far field signals. A near field signal is a signal that is detected by a sensing electrode(s) located in or immediately adjacent the same chamber as where the signal originates. A far field signal is a signal, that is detected by a sensing electrode(s) located in or immediately adjacent one chamber of the heart, but originated in another more remote chamber of the heart or outside of the heart. For example, the far field signal may originate in a chamber opposite to the chamber containing the sensing electrode(s) (e.g., opposite ventricle, or opposite atrium). As a further example, the RV lead 30 may detect near field signals originating in the right ventricle, as well as far field signals originating in the right atrium, the left ventricle, the left atrium or outside the heart. The LV lead 24 detects near field signals originating in the left ventricle, as well as far field signals originating in the right ventricle, left atrium and right atrium.

The device 10 utilizes a detection algorithm to analyze the near field signals for intrinsic heart activity. The device 10 utilizes an arrhythmia detection process to recognize arrhythmias, such as atrial fibrillation, flutter, bigeminy, trigeminy and the like. The device 10 senses the R-waves and preceding confirmation events in the near field and far field, respectively, over at least one channel. The device 10 may utilize a single channel, or may utilize separate primary and secondary channels. The single or primary channel may be defined between the RV shocking coil 36 and the RV tip electrode 32 (as denoted at 33) or between the RV tip electrode 32 and the RV ring electrode 34. Alternatively, the single channel or a secondary channel (denoted at 35) may be defined between the RV shocking coil 36 and the case of the device 10.

As explained below, the sensed signals are separated into near field signals that contain intrinsic R-wave events and far field signals that contain confirmation events. In accordance with at least one embodiment, the arrhythmia detection module identifies arrhythmia based on high rate intrinsic R-waves that are preceded by high rate confirmation events sensed over multiple (N) cardiac cycles. An intrinsic confirmation event may be one of a P-wave, a HIS signal and a PVC signal. In accordance with one embodiment, the device 10 further comprises a therapy control module that directs the pulse generator to provide a corrective therapy responsive to the arrhythmia detection module. Optionally, the therapy control module may deliver at least one ventricular pulse, as the corrective therapy.

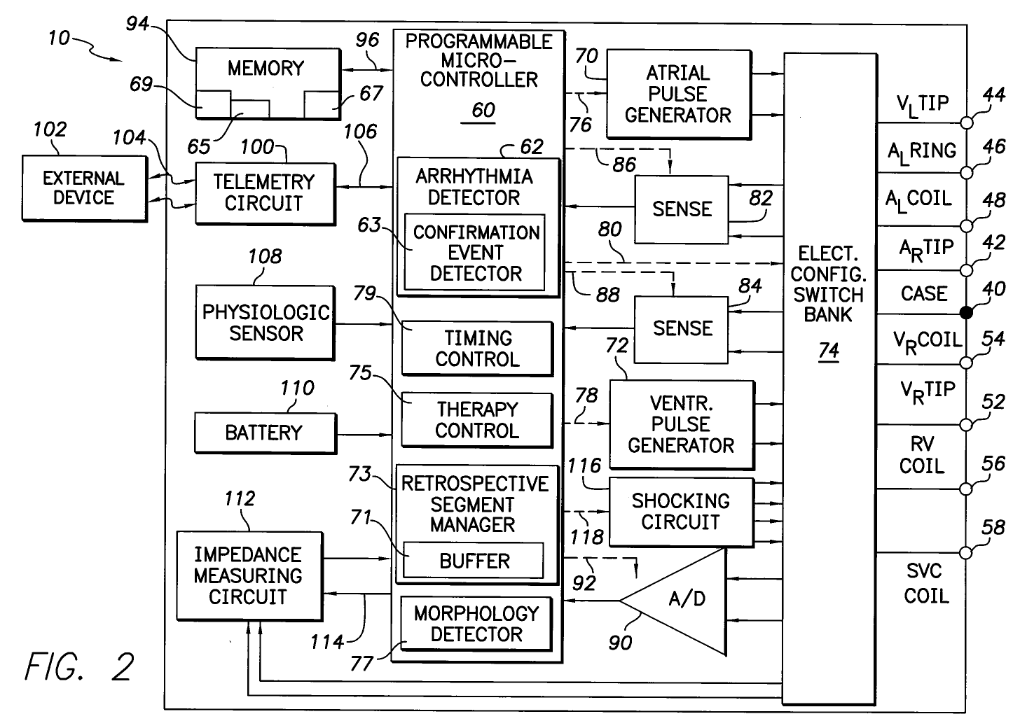

FIG. 2 illustrates a simplified block diagram of the implantable medical device 10, which is capable of treating both fast and slow arrhythmias with stimulation therapy, including cardioversion, defibrillation, and pacing stimulation. The device 10 may be a single chamber. While a particular device is shown, this is for illustration purposes only, and one of skill in the art could readily duplicate, eliminate or disable the appropriate circuitry in any desired combination to provide a device capable of treating the appropriate chamber with cardioversion, defibrillation and pacing stimulation. The blocks illustrated in FIG. 2 represent functional blocks which may be implemented in hardware, discrete logic, firmware, software, in or with a single CPU, multiple CPUs, field programmable gate arrays and the like. The terms “circuit” and “module” are used throughout interchangeably to refer to functional blocks.

The housing 40 for the device 10, shown schematically in FIG. 2, is often referred to as the “can”, “case” or “case electrode” and may be programmably selected to act as the return electrode for all “unipolar” modes. The housing 40 may further be used as a return electrode alone or in combination with one or more of the electrodes 36 and 38 for shocking purposes. The housing 40 further includes a connector (not shown) having a plurality of terminals, 42, 44, 46, 48, 52, 54, 56, and 58 (shown schematically and, for convenience, the names of the electrodes to which they are connected are shown next to the terminals).

The device may include a left ventricular tip terminal (VL TIP) 44, a left atrial ring terminal (AL RING) 46, and/or a left atrial shocking terminal (AL COIL) 48, which are adapted for connection to a left ventricular ring electrode, a left atrial tip electrode, and a left atrial coil electrode, respectively. To support right chamber sensing, pacing and shocking, the connector includes a right ventricular tip terminal (VR TIP) 52, a right ventricular ring terminal (VR RING) 54, a right ventricular shocking terminal (RV COIL) 56, and an SVC shocking terminal (SVC COIL) 58, which are adapted for connection to the right ventricular tip electrode 32, right ventricular ring electrode 34, the RV coil electrode 36, and the SVC coil electrode 38, respectively.

The device 10 includes a programmable microcontroller or processor 60 which controls the various modes of operation. As is well known in the art, the microcontroller 60 typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 60 includes the ability to process or monitor input signals (data) as controlled by a program code stored in a designated block of memory. The details of the design and operation of the microcontroller 60 are not critical to the present invention. Rather, any suitable microcontroller 60 may be used that carries out the functions described herein.

An atrial pulse generator 70 and a ventricular pulse generator 72 generate pacing stimulation pulses for delivery by the right atrial lead (not shown), the right ventricular lead 30, and/or a coronary sinus lead (not shown) via an electrode configuration switch 74. The atrial and ventricular pulse generators, 70 and 72, may include dedicated, independent pulse generators, multiplexed pulse generators, or shared pulse generators. The pulse generators, 70 and 72, are controlled by the microcontroller 60 via appropriate control signals, 76 and 78, respectively, to trigger or inhibit the stimulation pulses. The microcontroller 60 further includes timing control circuitry 79 which is used to control the timing of stimulation pulses (e.g., pacing rate) as well as to keep track of the timing of blanking intervals, noise detection windows, evoked response windows, alert intervals, marker channel timing, and the like.

The switch 74 includes a plurality of switches for connecting the desired electrodes to the appropriate I/O circuits, thereby providing complete electrode programmability. The switch 74, in response to a control signal 80 from the microcontroller 60, determines the polarity of the stimulation pulses (e.g., unipolar, bipolar, combipolar, etc.) by selectively closing the appropriate combination of switches (not shown).

Sensing circuits 82 and 84 are selectively coupled to different combinations of electrodes, such as the case 40 and on the right ventricular lead 30, through the switch 74 for detecting the presence of cardiac activity in the heart. The sensing circuits, 82 and 84, may include dedicated sense amplifiers, multiplexed amplifiers, or shared amplifiers. The switch 74 determines the “sensing polarity” of the cardiac signal by selectively closing the appropriate switches. In this way, the clinician may program the sensing polarity independent of the stimulation polarity. Each sensing circuit, 82 and 84, preferably employs one or more low power, precision amplifiers with programmable gain and/or automatic gain control, bandpass filtering, and a threshold detection circuit, to selectively sense the cardiac signal of interest. The automatic gain control enables the device 10 to sense the low amplitude signal characteristics of atrial or ventricular fibrillation.

The power, gain, filtering, threshold and other sensor parameters of the sensing circuits 82 and 84 may be predefined, programmable or adjustable by the microcontroller 60 through control lines 86 and 88. For example, the microcontroller 60 may set sensing circuit 82 to be less sensitive to far field signal components and more sensitive to near field signal components (e.g., by reducing the gain and/or shifting the cutoff frequencies of the band pass filter). The microcontroller 60 may set sensing circuit 84 to be less sensitive to near field signal components and more sensitive to far field signal components. Thus, the sensing circuit 82 passes, amplifies and separates the near field signal components and the sensing circuit 84 passes, amplifies and separates the far field signal components. The sensitivity of the sensing circuits 82 and 84 is controlled based in part on the locations of the sensors relative to the events of interest. For example, both of the sensing circuits 82 and 84 may be connected to the RV tip electrode 32 and RV coil electrode 36. The sensing circuit 82 may be controlled to be sensitive to events in the near field, namely in the right ventricle where both electrodes 32 and 36 are located. The sensing circuit 84 may be controlled to be sensitive to events in the far field, such as in the left ventricle or the right atrium which are remote from electrodes 32 and 36. As another example, the sensing circuit 82 may be connected to RV tip electrode 32 and RV coil electrode 36, while sensing circuit 84 is connected to the RV tip electrode 32 and the housing of the device 10. The sensing circuits 82 and 84 may still be controlled to be sensitive to events in the near field and far field, respectively.

In the exemplary embodiment, sensing circuits 82 and 84, switch 74 and their control by the microcontroller 60, are collectively referred to as a sensing module. In one embodiment, the sensing module defines a primary channel between a first combination or set of sensors and defines a secondary channel between a second combination or set of sensors. The first and second combinations of sensors may be identical or only partially overlapping or entirely mutually exclusive of one another.

The sensors may include at least one sensor pair that is remotely spaced from another pair. The primary channel may be coupled to low sensitivity amplifiers, and the secondary channel may be coupled to the high sensitivity amplifiers. The outputs of the sensing circuits, 82 and 84, are connected to the microcontroller 60 which, in turn, are able to trigger or inhibit the atrial and/or ventricular pulse generators, 70 and 72, respectively, in a demand fashion in response to the absence or presence of cardiac activity in the appropriate chambers of the heart.

By way of example, the primary channel may represent the near field in the region immediately adjacent to the RV coil or RV tip electrodes 34 and 32 or RV ring electrode 33. As a further example, the secondary channel may represent a far field channel that is configured to sense signals originating in a far field remote from the corresponding combination of sensors. The sensing module monitors the near field (NF) channel for intrinsic cardiac NF signal components originating within a chamber of the heart proximate the electrodes. The sensing module monitors the far field (FF) channel for intrinsic cardiac FF signal components originating within a chamber of the heart remote from the electrodes. For example, the sensing module may be configured to sense P-waves originating in the far field (e.g., in the right atrium) when detected at sensing vector 35 (FIG. 1) between the RV shocking coil and the case. Alternatively or in addition, the secondary channel may sense P-waves in the far field by defining a sensing vector between the case and the RV tip 32 or between the case and RV ring 33. As a further option, the far field sensing vector may be defined between the case and the SVC coil 38. As a further example, the secondary channel may be defined using one or more of sensing vector(s) between the RV coil and the SVC coil, between the RV tip and SV coil, and between the RV shocking coil and the SVC coil. The cardiac signals detected over the primary and secondary channels are delivered to the controller 60 for processing and analysis.

As a further option, when implemented in a remote monitoring device such as the Confirm™ device by St. Jude Medical, the far field sensing vector may be defined between two sensors located on the case of the monitoring device and spaced apart from one another. In this alternative embodiment, no lead may be provided within the heart.

The cardiac signals sensed in the far field may be passed to memory for long term or short term storage as far field signal components, such as to the buffer 71 or the memory 94. The cardiac signals sensed in the near field and/or over the primary channel are passed to the arrhythmia detector 62 to be analyzed as near field signal components.

As used herein “sensing” is reserved for the noting of an electrical signal, and “detection” is the processing of the sensed sequential signals and noting the presence of an event of interest and/or an arrhythmia. The timing intervals between sensed R-waves are then classified by the microcontroller 60 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.).

A confirmation event detector 63 seeks to identify intrinsic confirmation events, such as a P-wave. The confirmation event detector 63 may identify an intrinsic confirmation event based on several parameters, such as correlation to a template, the morphology of the event, comparison to past events and the like. A retrospective segment (RS) manager manages storage of far field cardiac signal components in a circular buffer 71. Optionally, the circular buffer 71 may be maintained in the memory 94. The buffer 71 is afforded a length that is sufficient to capture a desired number of cardiac cycles or a desired portion of a cardiac cycle. The buffer 71 is sized to correspond to the retrospective detection window 208 (e.g. 350 to 400 msec segment). The buffer 71 functions in a “circular” manner in that the RS manager 73 continuously writes newly received far field cardiac signals over the prior content of the buffer 71. Thus, at any point in time, the buffer 71 contains a snap-shot of the most recent far field cardiac signals sensed during the detection window 208.

Optionally, a morphology detector module 77 may be used to analyze shapes of cardiac signals. In accordance with one embodiment, the device 10 further comprises a therapy control module 75 that directs the shocking circuit 116 to provide a corrective therapy responsive to the arrhythmia detector module 62. Optionally, the therapy control module may deliver at least one atrial pulse and/or ventricular pulse, as the corrective therapy.

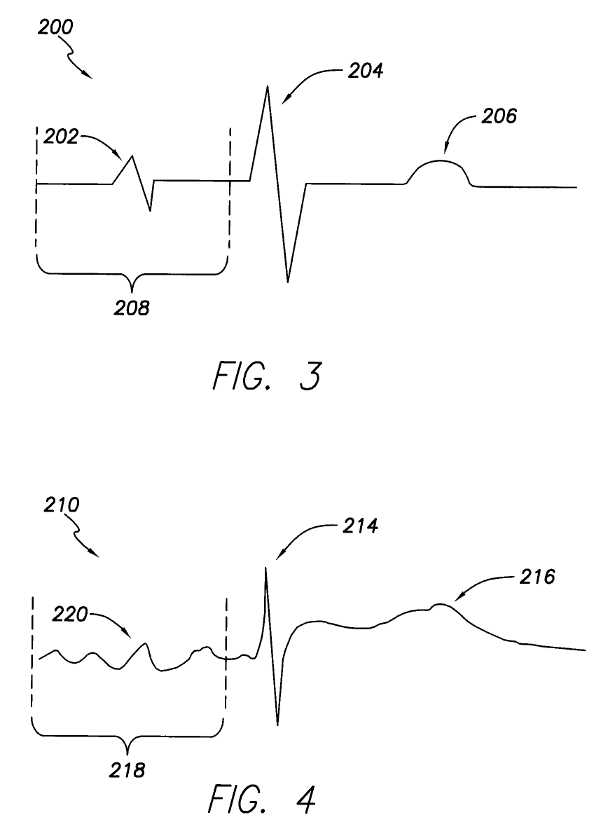

FIG. 3 illustrates an exemplary cardiac cycle that is representative of a physiologic or normal sinus rhythm. The cardiac cycle 200 in FIG. 3 includes a P-wave 202, an R-wave 204 and a T-wave 206. The R-waves are sensed in the near field of sensing vector 33 and/or over the primary channel (sensing vector 33) and the P-waves are sensed in the far field of sensing vector 33 and/or over the secondary channel (sensing vector 35). As explained below, the arrhythmia detector 62 identifies R-waves 204 occurring over multiple cardiac cycles 200 and analyzes the R-waves to determine whether a potential arrhythmia exists based upon the R to R interval, among other things. Before a series of R-waves 204 can be potentially representative of an arrhythmia, each of the R-waves should be preceded by a corresponding confirmation event.

In the example of FIG. 3, the confirmation events correspond to P-waves 202 that precede the corresponding R-waves 204. In FIG. 3, a retrospective detection window is denoted at 208. Optionally, the P-wave detection window may be rate dependent. The cardiac signals occurring during the retrospective detection window 208 are sensed and the far field signal components are obtained from sensing vector 33. Alternatively, the cardiac signals are sensed over the secondary channel (when present), such as along the sensing vector 35 (FIG. 1), in an effort to locate P-waves occurring in the far field. When a series of R-waves 204 are preceded by corresponding P-waves 202, the cardiac signals represent physiologic, normal sinus rhythms. When a series of high rate R-waves 204 are not preceded by corresponding P-waves, or the P-waves occur inconsistently, the cardiac signals represent an arrhythmia, such as AF.

FIG. 4 illustrates a cardiac cycle that would be sensed during an arrhythmia, in which a pathologic, abnormal sinus rhythm occurs. The pathologic cardiac cycle 210 includes an R-wave 214 and a T-wave 216, but does not include a normal P-wave. Instead, during the retrospective detection window 218, the cardiac signal 210 exhibits an unorganized signal pattern (denoted by pathologic signal 220). The confirmation event detector 63 and retrospective segment manager 73 seek to identify P-waves during the retrospective detection window 218. When, instead of a P-wave, a pathologic signal 220 is identified in combination with a high rate of R to R intervals, this behavior is a strong indicator that the series of R-to-R intervals are abnormal and warrant treatment.

Returning to FIG. 2, the cardiac signals are also applied to the inputs of an analog-to-digital (ND) data acquisition system 90. The data acquisition system 90 may receive control signals 92 and is configured to acquire intracardiac electrogram signals, convert the raw analog data into a digital signal, and store the digital signals for later processing and/or telemetric transmission to an external device 102. The data acquisition system 90 is coupled to the right ventricular lead 30 through the switch 74 to sample cardiac signals across any pair of desired sensor electrodes. Optionally, other leads with multiple electrodes may be added to the system, to further improve the diagnosis and characterization of the arrhythmia.

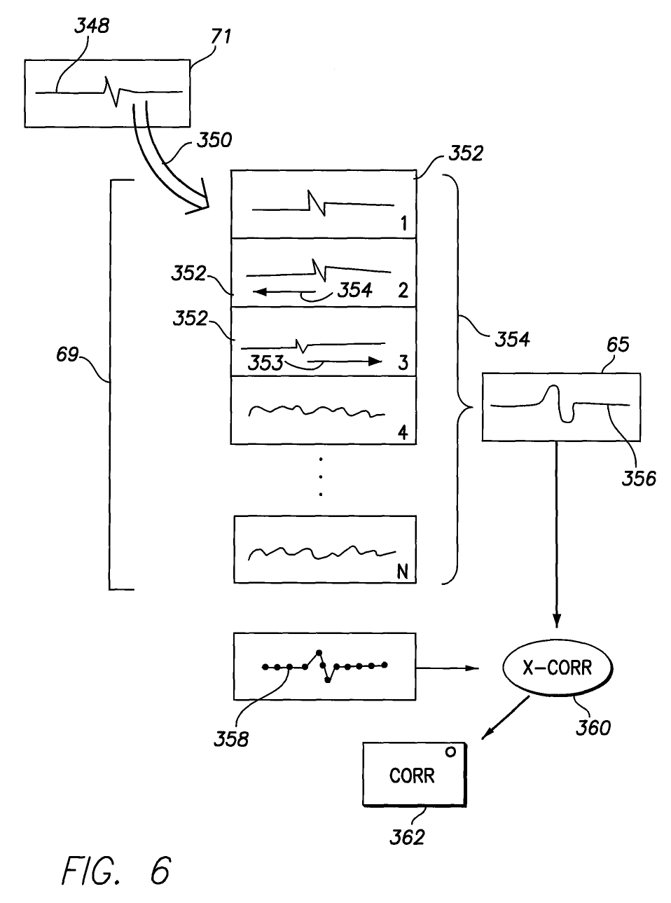

The microcontroller 60 is further coupled to a memory 94 by a suitable data/address bus 96, wherein the programmable operating parameters used by the microcontroller 60 are stored and modified, as required, in order to customize the operation of the stimulation device 10 to suit the needs of a particular patient. Such operating parameters define, for example, pacing pulse amplitude, pulse duration, PVARP interval, PVARP extension, PR threshold, RP threshold, R to R threshold, rate threshold, electrode polarity, rate, sensitivity, automatic features, arrhythmia detection criteria, and the amplitude, waveshape and vector of each shocking pulse to be delivered to the patient's heart 12 within each respective therapy. The memory 94 includes a log 67 and a memory stack 69 that stores the segments 352 (FIG. 6) of cardiac signals captured from the buffer 71. The memory 94 also includes memory 65 to save an ensemble segment 356 and a waveform template 358. The memory 94 may also store the number of corrective therapies to be delivered in successive cardiac cycles to attempt to correct an intrinsic reentrant tachycardia.

Advantageously, the operating parameters of the implantable device 10 may be non-invasively programmed into the memory 94 through a telemetry circuit 100 in telemetric communication with the external device 102, such as a programmer, transtelephonic transceiver, or a diagnostic system analyzer. The telemetry circuit 100 is activated by the microcontroller by a control signal 106. The telemetry circuit 100 advantageously allows intracardiac electrograms and status information relating to the operation of the device 10 (as contained in the microcontroller 60 or memory 94) to be sent to the external device 102 through an established communication link 104.

In one embodiment, the stimulation device 10 further includes a physiologic sensor 108, commonly referred to as a “rate-responsive” sensor because it is typically used to adjust pacing stimulation rate according to the exercise state of the patient. However, the physiological sensor 108 may further be used to detect changes in cardiac output, changes in the physiological condition of the heart, or diurnal changes in activity (e.g., detecting sleep and wake states). Accordingly, the microcontroller 60 responds by adjusting the various pacing parameters (such as rate, etc.) at which the atrial or ventricular pulse generator 70 or 72, generates stimulation pulses.

The device 10 additionally includes a battery 110 which provides operating power to all of the circuits shown in FIG. 2. For the device 10, which employs shocking therapy, the battery 110 is capable of operating at low current drains for long periods of time, and then be capable of providing high-current pulses (for capacitor charging) when the patient requires a shock pulse. The device 10 is shown as having an impedance measuring circuit 112 which is enabled by the microcontroller 60 via a control signal 114.

In the case where the device 10 is intended to operate as an implantable cardioverter/defibrillator (ICD) device, it must detect the occurrence of an arrhythmia, and automatically apply an appropriate electrical shock therapy to the heart aimed at terminating the detected arrhythmia. To this end, the microcontroller 60 further controls a shocking circuit 116 by way of a control signal 118. The shocking circuit 116 generates shocking pulses of low (up to 5 joules), moderate (6 to 15 joules), or high energy (16 to 40 joules), as controlled by the microcontroller 60. Cardioversion shocks are generally considered to be of low to moderate energy level and/or synchronized with an R-wave and/or pertaining to the treatment of tachycardia. Defibrillation shocks are generally of moderate to high energy level, i.e., corresponding to outputs in the range of 16-40 joules. Although external ICDs deliver the shock asynchronously (since R-waves may be too disorganized and small) in the setting of ventricular fibrillation, the implantable devices still synchronize with a ventricular depolarization signal as fibrillatory signals as recorded from inside the heart may be very discrete.

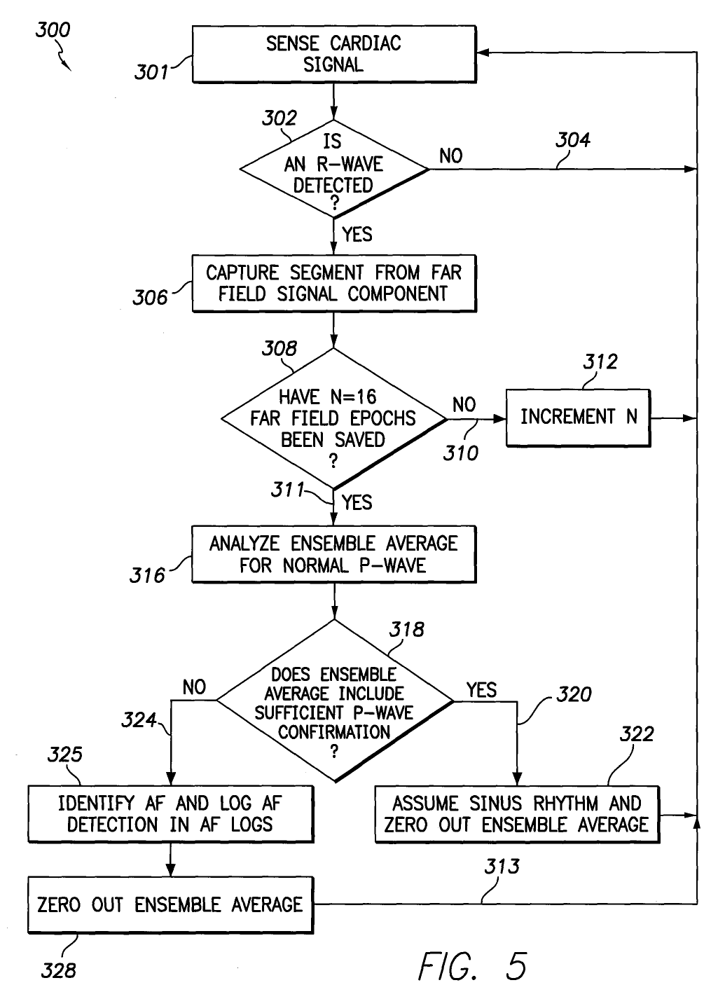

FIG. 5 illustrates a flow chart showing the processing sequence to detect an arrhythmia based on the absence or inconsistent confirmation events in accordance with an embodiment of the present invention. The process 300 may be carried out by various external or implanted devices or systems, such as described herein. The process 300 may be applied at all times or only whenever a potential elevated ventricular rate is detected. At 301, cardiac signals are sensed and recorded. For example, the cardiac signals may include atrial activity for a confirmation event, such as a P-wave in the far field. Alternatively, the cardiac signals may include a confirmation event that represents a HIS signal or a premature ventricular contraction (PVC) signal. At least the far field cardiac signals are stored in the circular buffer 71 under control of the retrospective segment manager 73.

By way of example, a secondary channel may be used that corresponds to a sensing vector sensitive to far field signals. The secondary channel may be between i) the RV shocking coil and the case, ii) the RV ring and case, iii) the RV tip and case, iv) the SVC coil and case, or v) the RV shocking coil and case. The secondary channel may also be defined between i) the RV ring and SVC coil, ii) the RV tip and SVC coil, or iii) the RV shocking coil and SVC coil. A primary channel may be used that corresponds to a sensing vector sensitive to near field signals. The primary channel may be between the RV shocking coil and the RV tip or RV ring and RV tip.

At 302, the near field cardiac signal component is analyzed to determine whether an R-wave is present. If an R-wave is not detected, then flow returns along path 304 until an R-wave is detected. When an R-wave is detected, then at 306, the RS manager 73 captures the current segment of the far field cardiac signal component which may be sensed over the same channel as the R-wave or over the secondary channel during the detection window 208 (FIG. 3). The current segment captured in the buffer 71 represents a “retrospective” segment in that the segment contains the portion of the cardiac signal, detected in the far field, immediately preceding the R-wave detected in the near field. The current segment is captured by moving the segment from the buffer 71 to a memory stack 69 in memory 94. The memory stack 69 may be organized to store a predetermined number of retrospective segments.

The RS manager 73 may manage the memory stack 69 also in a circular manner such that the newest segment, written to the memory stack 69, is written over the oldest segment on the memory stack 69. Alternatively, the RS manager 73 may load the memory stack 69 in batches such that the memory stack 69 is filled with retrospective segments and then entirely dumped/cleared once a sufficient number of retrospective segments have been written to the memory stack 69, before beginning to refill the memory stack 69.

Optionally, the length of the buffer 71 and memory stack 69 may be adjusted to modify the length of the detection window 208 of the cardiac signal that is stored during each retrospective segment. In the exemplary embodiment, an interval of 350 msec is utilized. However, the interval may be longer or shorter, for example 500 msec, 300 msec or otherwise. As a further example, a normal P-wave interval may occur up to 150 msec before the occurrence of a corresponding succeeding R-wave. Hence, the length of the buffer 71 and memory stack 69 may equal 150 msecs or more.

Optionally, the number of segments 352 maintained in the memory stack 69 may be programmable. For example, the number of segments 352 in the memory stack 69 may include 16 retrospective segments. Alternatively, fewer or more than 16 retrospective segments may be utilized.

Next, at 308, it is determined whether a predetermined number N of retrospective segments has been captured and moved to the memory stack 69. Each retrospective segment corresponds to an event confirmation window or epoch. If the desired number of event confirmation windows has not yet been saved, then the segment counter is incremented at 312 and flow returns along path 310 to 301. The process repeats operations 301 to 312 until N segments are captured and moved to the memory stack 69. When a desired number of segments have been saved in the memory stack 69, flow moves along path 311 to 316.

FIG. 6 illustrates a graphical representation of how the RS manager 73 and confirmation event detector 63 manage and analyze retrospective segments. The RS manager 73 continuously fills the circular buffer 71 with cardiac signals detected in the far field. The cardiac signal stored in the buffer 71 represents a portion of an intercardiac electrogram (IEGM) 348. Once an R-wave is detected in the near field at 302 (FIG. 5), the cardiac signal from the buffer 71 is moved along path 350 to the memory stack 69. The memory stack 69 includes a series of memory addresses that are each apportioned to store one segment 352. The RS manager 73 continuously moves segments from the buffer 71 onto the memory stack 69, during each cardiac cycle for which an R-wave is detected, until filling the memory stack 69.

Returning to FIG. 5, during 306 and 308, the RS manager 73 combines the plurality of retrospective segments stored in the memory stack 69 to form the segment ensemble 356 (FIG. 6). The retrospective segments may be combined in different manners. For example, the retrospective segments may be averaged with one another to form the segment ensemble. Alternatively, a mean or median of the retrospective segments may be utilized as the segment ensemble. The RS manager 73 may combine each new retrospective segment with a running average. Alternatively, the RS manage 73 may wait until the number N of retrospective segments is captures and then combine the group of N individual retrospective segments. In the latter example, each segment ensemble would be formed from a different non-overlapping group of N retrospective segments, whereas in the former example, the segment ensemble would reflect a running average partially representative of retrospective segments that occurred before the N most recent retrospective segments.

At 316, the ensemble average is analyzed for normal P-wave activity in the far field. For example, the confirmation event detector 63 may cross correlate 360 (FIG. 6) the segment ensemble 356 of retrospective segments to a P-wave template or to a prior normal actual P-wave 358 to obtain a degree of correlation 362 there between. Optionally, at 316, ensemble averages (EA) may be analyzed by comparing a current EA with one or more prior EA. Alternatively, a current EA may be compared to an average of prior EAs. Alternatively, the EA may be analyzed based on the root mean square of one or more EAs.

Optionally, prior to the correlation 360, the confirmation event detector 63 may perform an alignment operation for each of the segments 352 in the memory stack 69. For example, to align the segments 352, the morphology or peak within each segment 352 may be analyzed and the waveforms within segments 352 may be shifted forward 353 or backward 354 until the peaks across the segments 352 align with one another. Once the segments 352 are aligned based upon their peak content or morphology, then the segment ensemble 356 may be created.

Optionally, the segments 352 may not be aligned with one another, but instead maintained in their original captured temporal state relative to one another without any time shifting therebetween. As a further option, once the segment ensemble 356 is determined, the segment ensemble 356 may be stored as the new waveform template 358 to replace the prior waveform template. In one embodiment, the waveform template 358 may be preprogrammed and maintained separate and apart from the individual physiology of a particular patient. Alternatively, the waveform template 358 may be obtained during a calibration operation by the device to tailor the waveform temple (e.g., the P-wave) to the particular morphology of an individual patient. As a further option, the waveform template 358 may be updated throughout operation, such as by replacement with the segment ensemble 356, each time a segment ensemble 356 is determined to closely correlate to a P-wave or other confirmation event.

Optionally, the coefficient threshold may be increased or decreased or may be set at a programmable level by a physician. When increasing or decreasing the coefficient threshold it is appreciated that adjusting this threshold increases or decreases the likelihood of false positives. By changing the threshold or the degree of correlation between the segment ensemble and the template, the user is requiring more or less correlation between the segment ensemble and the template. Hence, by decreasing the threshold, less correlation is acceptable which may increase the likelihood that a particular event is determined to be physiologic (e.g., normal sinus rhythm) and not pathologic, when in fact the underlying cardiac signal did not include a confirmation event and therefore should have been characterized as an arrhythmia.

Alternatively, when the coefficient threshold is increased, a greater degree of correlation is required. When the coefficient threshold is increased significantly, the situation may arise in which a retrospective segment includes a valid confirmation event thereby indicating that the cardiac signal is physiologic, yet the device finds an insufficient level of correlation and declares the event to not be physiologic and to be an arrhythmia. The physician may change the coefficient threshold dependent upon the desired level of specificity and the desired percentage of the time that false positives are acceptable to be declared.

At 318, it is determined whether the ensemble average includes sufficient P-wave confirmation. The confirmation event detector 63 may identify the arrhythmia based at least in part on the degree of correlation. For example, the confirmation event detector 63 determines whether the degree of correlation exceeds the coefficient threshold. The coefficient threshold may be programmed by a physician. Alternatively, the coefficient threshold may be adjusted throughout operation of the device 10 based upon prior detected cardiac signals. When the degree of correlation exceeds the coefficient threshold, the segment ensemble is determined to represent an intrinsic confirmation event. When an intrinsic confirmation event is detected, flow moves along path 320. At 322, the confirmation event detector 63 clears or zeros the segment ensemble stored in the memory stack 69. Thereafter, flow returns to the beginning of the process 300.

Returning to 318, when the degree of correlation falls below the correlation coefficient threshold, the segment ensemble is determined to lack an intrinsic confirmation event. When an intrinsic confirmation event(s) is absent from the segment ensemble, the confirmation event detector 63 determines that the R-wave event(s) detected are not representative of a normal sinus rhythm. When the R-wave event(s) are not representative of a normal sinus rhythm, flow moves along path 324.

As a further option, the operations at 316 and 318 (FIG. 5) may be modified, such that each individual retrospective segment 352 is compared to the waveform template 358. In this alternative implementation, an ensemble average is not generated. Instead, the waveform template 358 is compared to each individual retrospective segment 352 within the memory stack 69. The confirmation event detector 63 maintains a count of the number of retrospective segments 352 that when correlated to the waveform template 358 satisfy the correlation coefficient threshold. When a sufficient number of the segments 352 out of a programmable group are determined to correlate with the waveform template 358, the confirmation event detector 63 declares the segments to be indicative of intrinsic confirmation events. For example, when 10 out of 16 segments 352 correlate with the template 358, the group of 16 R-waves are considered to have normal sinus rhythm. Optionally, AF may be declared when 7 out of 20 segments lack a predetermined amount of correlation to the template 358.

At 326, the arrhythmia detector 62 identifies the type and nature of the arrhythmia and records indicators for the arrhythmia in a log 67 in memory 94. The arrhythmia indicators may include the time at which the arrhythmia occurred, the duration of the arrhythmia, the nature or type of the arrhythmia, the arrhythmia burden and the like. At 328, the segment ensemble is cleared or zeroed in the memory stack 69. Thereafter, the flow returns along 313 to the beginning of the process 300.

Optionally, the gain of the far field signal may be increased to further amplify the far field P-wave. A further enhancement of the signal to noise ratio is achieved by ensemble averaging several buffered, retrospective windows preceding the R-wave to create a signal averaged P-wave. The signal to noise ratio is increased by the square root of the number of P-waves included in the signal averaged P-wave. Optionally, the device 10 may vibrate or emit an audible alarm when an arrhythmia is declared.

The flow chart of FIG. 5 describes an overview of the operation as implemented in one embodiment of the device 10. In this flow chart the various algorithmic steps are summarized in individual “blocks”. Such blocks describe specific actions or decisions made or carried out as the algorithm proceeds. Where a microcontroller (or equivalent) is employed, the flow charts presented herein provide the basis for a “control program” that may be used by such a microcontroller (or equivalent) to effectuate the desired control of the stimulation device. Those skilled in the art may readily write such a control program based on the flow charts and other descriptions presented herein.

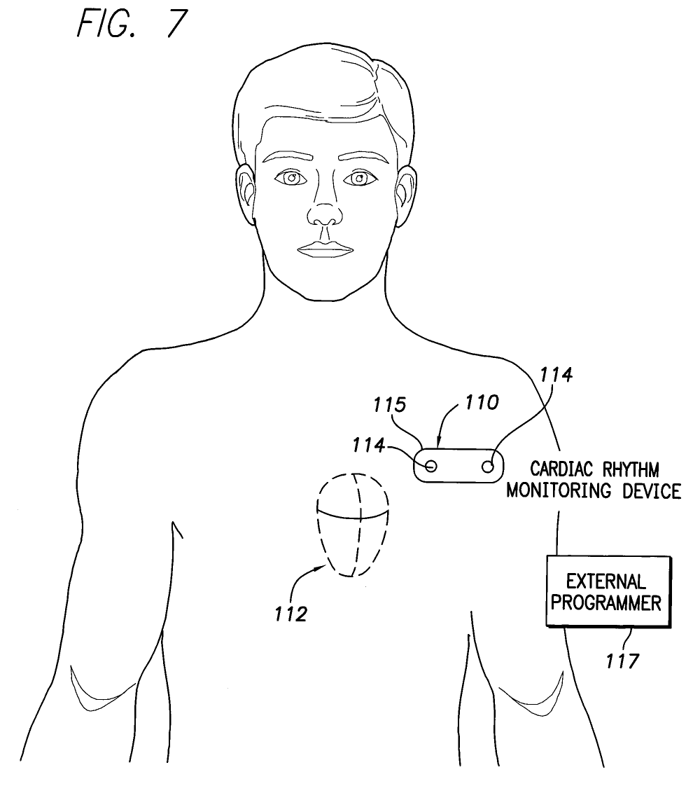

FIG. 7 illustrates an implantable monitoring device 110 intended for subcutaneous implantation at a site near the heart 12. The monitoring device includes a pair of spaced-apart sense electrodes 114 positioned with respect to a housing 115. The sense electrodes 114 provide for detection of far field electrogram signals. Numerous configurations of electrode arrangements are possible. For example, the electrodes 114 may be located on the same side of the housing 115. Alternatively, the electrodes 114 may be located on opposite sides of the housing 115. One of the electrodes 114 may be formed as part of the housing 115, for example, by coating all but a portion of the housing with a nonconductive material such that the uncoated portion forms the electrode. In this case, the other of the electrodes 114 may be electrically isolated from the housing electrode by placing it on a component separate from the housing, such as a header (not shown). In other configurations, the electrodes 114 may be located on short, stub leads extending away from the housing but coupled thereto through one or more headers so as to interface with internal components. The housing 115 includes various other components such as: sense module for receiving signals from the sensors, a microprocessor for processing the signals in accordance with algorithms, such as the AF detection algorithm described herein, a loop memory for temporary storage of electrograms, a device memory for long-term storage of electrograms upon certain triggering events, such as AF detection, sensors for detecting patient activity and a battery for powering components.

The monitoring device 110 performs the arrhythmia detection process as set forth in connection with FIGS. 2-6. The monitoring device 110 senses far field electrograms; processes the electrograms to detect arrhythmias and if an arrhythmia is detected; automatically records the electrograms in memory for a subsequent transmission to an external device 117. Electrogram processing and arrhythmia detection is provided for, at least in part, by algorithms embodied in the microprocessor. In one configuration, the monitoring device is operative to detect atrial fibrillation.

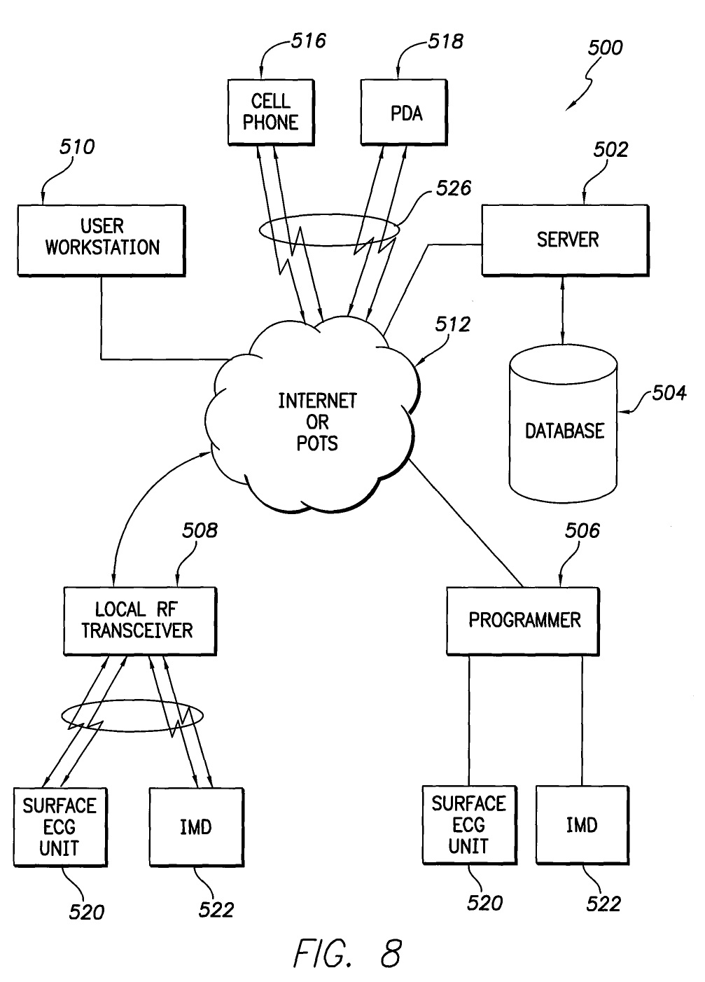

FIG. 8 illustrates a distributed processing system 500 in accordance with one embodiment. The distributed processing system 500 includes a server 502 that is connected to a database 504, a programmer 506, a local RF transceiver 508 and a user workstation 510 electrically connected to a communication system 512. The system 500 may be used to support remote monitoring of devices 10 (noted as IMDs 522). For example, when a device 10 declares an arrhythmia, the device 10 may transmit a message to the local RF transceiver 508 or programmer 506 which passes the message to a physician's workstation, PDS, cell phone, etc. The device 10 may transmit a notice to a physician when AF burden occurs a predetermined number of times or a predetermined amount of time in one day or one week. The server 502 may keep records to determine where to route an AF notice (e.g., which IP address, which physician, etc.).

The communication system 512 may be the internet, a voice over IP (VoIP) gateway, a local plain old telephone service (POTS) such as a public switched telephone network (PSTN), and the like. Alternatively, the communication system 512 may be a local area network (LAN), a campus area network (CAN), a metropolitan area network (MAN), or a wide area network (WAM). The server 502 interfaces with the communication system 512, such as the Internet or a local POTS based telephone system, to transfer information between the programmer 506, the local RF transceiver 508, the user workstation 510 as well as a cell phone 516, and a personal data assistant (PDA) 518 to the database 504 for storage/retrieval of records of information. For instance, the server 502 may download, via a wireless connection 526, to the cell phone 516 or the PDA 518 the results of processed cardiac signals, ST segment trends, impedance vectors, or a patient's physiological state (e.g., is the patient having or has had an arrhythmia) based on previously recorded cardiac information. On the other hand, the server 502 may upload raw cardiac signals (e.g., unprocessed cardiac data) from a surface ECG unit 520 or an IMD 522 via the local RF transceiver 508 or the programmer 506.

Database 504 is any commercially available database that stores information in a record format in electronic memory. The database 504 stores information such as, arrhythmia information, burden, retrospective segments, segment ensembles, waveform templates, raw cardiac data, processed cardiac signals, statistical calculations (e.g., averages, modes, standard deviations), histograms, cardiac trends (e.g., STS trends), and the like. The information is downloaded into the database 504 via the server 502 or, alternatively, the information is uploaded to the server from the database 504.

The Programmer 506 interfaces with the surface ECG unit 520 and the IMD 522 (e.g., similar to the device 10 described above and shown in FIG. 1). The programmer 506 may wirelessly communicate with the IMD 522 and utilize protocols, such as Bluetooth, GSM, infrared wireless LANs, HIPERLAN, 3 G, satellite, as well as circuit and packet data protocols, and the like. The programmer 506 is able to acquire cardiac signals from the surface of a person (e.g., ECGs), or the programmer is able to acquire intra-cardiac electrogram (e.g., IEGM) signals, arrhythmia information, burden, retrospective segments, segment ensembles and waveform templates, from the IMD 522. The programmer 506 interfaces with the communication system 512, either via the internet or via POTS, to upload the cardiac data acquired from the surface ECG unit 520 or the IMD 522 to the server 502. The programmer 506 may upload more than just raw cardiac data. For instance, the programmer 506 may upload status information, operating parameters, therapy parameters, patient status, access settings, software programming version, ST segment thresholds, calculated or measured impedance vectors, and the like.

The user workstation 510 may interface with the communication system 512 via the internet or POTS to download information via the server 502 from the database 504. Alternatively, the user workstation 510 may download raw data from the surface ECG unit 520 or IMD 522 via either the programmer 506 or the local RF transceiver 508. Once the user workstation 510 has downloaded the cardiac information (e.g., raw cardiac signals, arrhythmia information, burden, retrospective segments, segment ensembles, waveform templates, ST segments, impedance vectors, and the like), the user workstation 510 may process the cardiac signals, create histograms, calculate statistical parameters, or determine cardiac trends and determine if the patient is suffering from ischemia or another physiological condition. Once the user workstation 510 has finished performing its calculations, the user workstation 510 may either download the results to the cell phone 516, the PDA 518, the local RF transceiver 508, the programmer 506, or to the server 502 to be stored on the database 504.

While the invention has been described by means of specific embodiments and applications thereof, it is understood that numerous modifications and variations may be made thereto by those skilled in the art without departing from the spirit and scope of the invention.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the timing dimensions, configurations and components described herein are intended to define parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

What is claimed is:

1. An implantable device, comprising:
a housing;
sensors configured to be located proximate to a heart;
a sensing module to sense cardiac signals originating from the heart over a channel defined by the sensors, the cardiac signals including intrinsic R-wave events and associated intrinsic confirmation events when the heart exhibits normal sinus rhythm;
memory to store the cardiac signals sensed over the channel; and
a detection module to identify an R-wave event within the cardiac signals, the detection module to capture, in the memory, a segment of the cardiac signals that precedes the R-wave event as a retrospective segment, the detection module to determine whether the retrospective segment includes an intrinsic confirmation event that is associated with and occurs before the R-wave event, the detection module to declare an arrhythmia based at least in part on the determination of whether the intrinsic confirmation event is absent from the retrospective segment.

2. The device of claim 1, further comprising a lead configured to be coupled to the housing and to be located proximate to a heart, the lead including at least a portion of the sensors utilized to define a primary channel and a secondary channel.

3. The device of claim 1, wherein the cardiac signals include a near field signal component and a far field signal component, the detection module identifying the R-wave event from the near field signal component, the detection module analyzing the far field signal component for the intrinsic confirmation event.

4. The device of claim 1, wherein the sensing module utilizes different sets of sensors to sense cardiac signals over primary and secondary channels.

5. The device of claim 1, wherein the sensing module includes high and low sensitivity amplifiers coupled to the sensors, the high sensitivity amplifiers separating a far field signal component, the low sensitivity amplifiers separating a near field signal component.

6. The device of claim 1, wherein the intrinsic confirmation event is one of a P-wave, a HIS signal and a PVC signal.

7. The device of claim 1, wherein the detection module declares the arrhythmia to constitute one of atrial fibrillation, flutter, and bigeminy.

8. The device of claim 1, wherein the sensing module monitors a near field (NF) channel for the R-wave event originating within a chamber of the heart proximate to a first sensor and monitors a far field (FF) channel for the intrinsic confirmation event originating within a chamber of the heart remote from the first sensor.

9. The device of claim 1, the detection module to combine a plurality of retrospective segments to form an ensemble average of retrospective segments.

10. The device of claim 9, wherein the detection module compares current and prior ensemble averages in connection with declaring the arrhythmia.

11. The device of claim 1, wherein the detection module correlates multiple retrospective segments to obtain a degree of correlation, the detection module to identify the arrhythmia based at least in part on the degree of correlation.

12. The device of claim 1, wherein the sensors are provided on the housing and on a lead to define a secondary channel to sense a far field signal component that includes the intrinsic confirmation event when in normal sinus rhythm.

13. The device of claim 1, wherein the sensors include a sensor pair that is remotely spaced from one another, the sensing module utilizing the sensor pair to define a secondary channel.

14. The device of claim 1, wherein the sensors include at least one of an RV shocking coil, RV ring, an SVC coil and RV tip, the sensing module defining a secondary channel between one of the SVC and the housing and one of the RV shocking coil, RV ring, and RV tip.

15. A method for confirming an arrhythmia in an implantable device, comprising:
sensing cardiac signals originating from the heart over a channel defined by sensors that are configured to be located proximate a heart;
storing, in memory in the implantable device, the cardiac signals sensed, the cardiac signals including intrinsic R-wave events and associated intrinsic confirmation events when the heart exhibits normal sinus rhythm;
identifying an R-wave event within the cardiac signals sensed over the channel;
when the R-wave event is identified, capturing a segment of the cardiac signals sensed that precedes the R-wave event as a retrospective segment;
determining whether the retrospective segment includes an intrinsic confirmation event that is associated with and occurs before the R-wave event identified; and
declaring an arrhythmia based, at least in part, on the determination of whether the intrinsic confirmation event is absent from the retrospective segment.

16. The method of claim 15, further comprising coupling a lead to the housing and locating the lead proximate to the heart, the lead including at least a portion of the sensors utilized to define a primary channel and a secondary channel.

17. The method of claim 15, wherein the cardiac signals include a near field signal component and a far field signal component, the method further comprising identifying the R-wave event from the near field signal component and analyzing the far field signal component for the intrinsic confirmation event.

18. The method of claim 15, further comprising utilizing different sets of sensors to sense cardiac signals over primary and secondary channels.

19. The method of claim 15, wherein the intrinsic confirmation event is one of a P-wave, a HIS signal and a PVC signal.

20. The method of claim 15, wherein the declaring includes declaring the arrhythmia to be one of atrial fibrillation, flutter, and bigeminy.

21. The method of claim 15, wherein the sensing includes monitoring a near field (NF) channel for the R-wave event originating within a chamber of the heart proximate to a first sensor, and monitoring a far field (FF) channel for the intrinsic confirmation event originating within a chamber of the heart remote from the sensor.

22. The method of claim 15, wherein the identifying identifies the R-wave event over multiple cardiac cycles.
